(19) United States
(12) Patent Application Publication
(10) Pub. No.: US 2012/0101302 A1
(43) Pub. Date: Apr. 26, 2012

(54) NOVEL PRECURSOR MOLECULES FOR F-18 LABELLED PET TRACERS

(75) Inventors: Georg Kettschau, Berlin (DE); Tobias Heinrich, Berlin (DE); Lutz Lehmann, Berlin (DE); Stephan Siegel, Berlin (DE); Sangram Nag, Stockholm - Kista (SE)

(73) Assignee: Bayer Pharma Aktiengesellschaft, Berlin (DE)

(21) Appl. No.: 13/265,068

(22) PCT Filed: Apr. 10, 2010
(86) PCT No.: PCT/EP2010/002237

§ 371 Date: Dec. 20, 2011

(30) Foreign Application Priority Data
Apr. 23, 2009 (EP) 09075190.0

(51) Int. Cl. C07C 211/29; C07C 209/74

(52) U.S. Cl. 564/366

(57) ABSTRACT

This invention relates to novel compounds suitable as precursors for the preparation of certain F-18 labeled positron emission tomography (PET) tracers. Furthermore, the invention relates to the preparation of such precursor molecules and to the preparation of PET tracers by F-18 labeling of such precursors.

### FIELD OF THE INVENTION

This invention relates to novel compounds suitable as precursors for the preparation of certain F-18 labelled positron emission tomography (PET) tracers. Furthermore, the invention relates to the preparation of such precursor molecules and to the preparation of PET tracers by F-18 labelling of such precursors.

### BACKGROUND

Molecular imaging has the potential to detect disease progression or therapeutic effectiveness earlier than most conventional methods in the fields of oncology, neurology and cardiology. Of the several promising molecular imaging technologies having been developed such as optical imaging, MRI, SPECT and PET, PET is of particular interest for drug development because of its high sensitivity and ability to provide quantitative and kinetic data.

For example positron emitting isotopes include carbon, iodine, fluorine, nitrogen, and oxygen. These isotopes can replace their non-radioactive counterparts in target compounds to produce tracers that function biologically and are chemically identical to the original molecules for PET imaging, or can be attached to said counterparts to give close analogues of the respective parent effector molecule. Among these isotopes ¹⁸F is the most convenient labelling isotope due to its relatively long half life (110 min) which permits the preparation of diagnostic tracers and subsequent study of biochemical processes. In addition, its low β+ energy (634 keV) is also advantageous.

The nucleophilic aromatic and aliphatic [¹⁸F]-fluoro-fluorination reaction is of great importance for [¹⁸F]-fluoro-labelled radiopharmaceuticals which are used as in vivo imaging agents targeting and visualizing diseases, e.g. solid tumours or diseases of brain. A very important technical goal in using [¹⁸F]-fluoro-labelled radiopharmaceuticals is the quick preparation and administration of the radioactive compound.

Monoamine Oxidases (MAO, EC, 1.4.3.4) is a distinct class of amine oxidases. MAO is present in two forms: MAO A and MAO B (Med. Res. Rev. 1984, 4, 323-358). Crystal structures of MAO A and MAO B complexed by ligands have been reported (J. Med. Chem. 2004, 47, 1767-1774 and Proc. Nat. Acad. Sci. USA, 2005, 102, 12684-12689).

Inhibitors that are selective for either isozyme have been identified and investigated (e.g. J. Med. Chem. 2004, 47, 1767-1774 and Proc. Nat. Acad. Sci. USA, 2005, 102, 12684-12689). Deprenyl (A) (Biochem Pharmacol. 1972, 5, 393-408) and clorgyline (B) are potent inhibitors of mono amine oxidase inducing irreversible inhibition of the enzymes. The L-isomer of deprenyl (C) is a more potent inhibitor than the D-isomer.

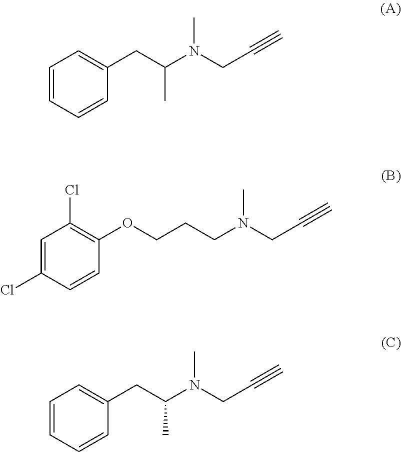

Neuroprotective and other pharmaceutical effects have also been described for MAO inhibitors (Nature Reviews Neuroscience, 2006, 295, 295-309, Br. J. Pharmacol., 2006, 147, 5287-5296). MAO B inhibitors are for example used to increase DOPA levels in CNS (Progr. Drug Res. 1992, 38, 171-297) and they have been used in clinical trials for the treatment of Alzheimer's disease based on the fact that an increased level of MAO B is involved in astrocytes associated with Alzheimer plaques (Neuroscience, 1994, 62, 15-30).

Fluorinated MAO inhibitors have been synthesised and biochemically evaluated (Kirk et al., Fluorine and Health, A. Tressaud and G. Haufe (editors), Elsevier 2008, pp 662-699). F-18 and C-11 labelled MAO inhibitors have been studied in vivo (Journal of the Neurological Science, (2007), 255, 17-22; review: Methods 2002, 27, 263-277). F-18 labelled deprenyl and deprenyl analogues (D) and (E) have also been reported (int. J. Radiat. Appl. instrument. Part A, Applied Radiat isotopes, 1991, 42, 121, J. Med. Chem. 1990, 33, 2015-2019 and Nucl. Med. Biol. 1990, 26, 111-116, respectively).

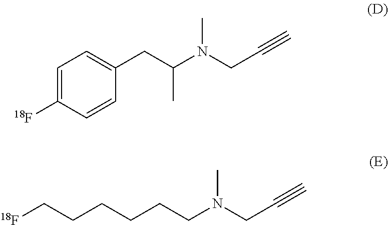

Patent application WO 2009/052970 inter alia teaches the application of isomeric mixtures such as shown in Scheme 1 (structures I and II) for the preparation of certain compounds useful for the diagnosis of diseases of the CNS, in particular those associated with increased levels of monoamine oxidase (MAO). Structurally somewhat related compounds are known to readily undergo rearrangements involving an intermediate aziridinium ion (see e.g. P. Gmeiner et al., J. Org. Chem. 1994, 59, 6766), leading to the formation of pure analogues of II under very mild conditions by means of rearrangement of the kinetically controlled analogues of I to the thermodynamically more stable analogue of II.

We found this rearrangement applicable for said isomeric mixture disclosed in WO 2009/052970 to give an almost pure regioisomer of the formula II. Surprisingly, and despite the fact that the steric demand of the nitrogen substitution is apparently lower in the compounds disclosed herein as compared to the system described in the reference above, this required substantially harsher conditions (heating to a temperature range of 70° C. to 130° C., preferred 80° C. to 120° C., even more preferred 90° C. to 110° C., instead of stirring at room temperature).

Utilisation of a regioisomerically pure compound II as shown in Scheme 1 offers the advantage of facilitated characterisation, quality control and regulatory proceedings as compared to the regioisomeric mixture disclosed in WO 2009/052970. In addition, the compounds of the general formula II features a much higher stability as compared to compounds of the general formula I. Thus, surprisingly the utilisation of a secondary precursor of the general formula II for a radiotracer of the general formula If is more advantageous as the use of the structurally much more related primary precursor of the general formula I.

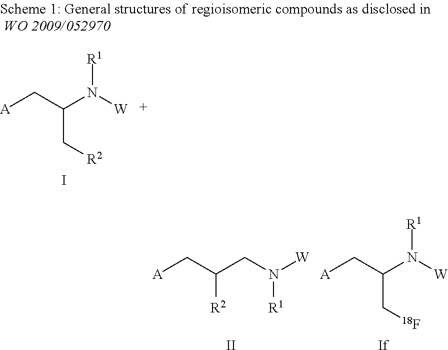

### DETAILED DESCRIPTION OF THE INVENTION

In a first aspect the invention is directed towards compounds of general formulae I and II

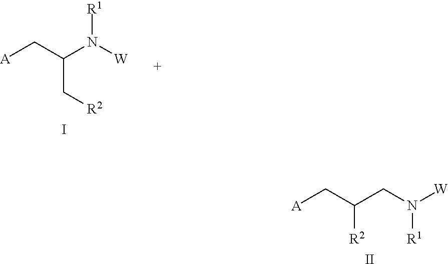

wherein

W is selected from the group comprising

—C(U¹)(U²)—C≡CH and cyclopropyl, U¹ and U² being independently selected from hydrogen and deuterium,

A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,

R¹ is selected from (C₁-C₆)-alkyl, preferably methyl,

R² is a leaving group, wherein preferred leaving groups are selected from halogen, C₁-C₆-alkylsulphonyloxy, which is optionally substituted by fluorine, and arylsulphonyloxy, which is optionally substituted by hydrogen, methyl, halo and nitro, and wherein particularly preferred leaving groups are chloro, bromo, methanesulphonyloxy, and p-toluenesulphonyloxy,

including all stereoisomeric forms of said compounds, including but not limited to enantiomers and diastereoisomers as well as racemic mixtures,

and any suitable salt with an organic or inorganic acid, ester, complex or solvate thereof.

In one embodiment, the invention is directed towards a compound of the general formula II, wherein

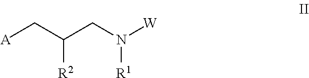

W is selected from the group comprising

—C(U¹)(U²)—C≡CH and cyclopropyl, U¹ and U² being independently selected from hydrogen and deuterium,

A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,

R¹ is selected from (C₁-C₆)alkyl, preferably methyl,

R² is a leaving group, wherein preferred leaving groups are selected from halogen, C₁-C₆-alkylsulphonyloxy, which is optionally substituted by fluorine, and arylsulphonyloxy, which is optionally substituted by hydrogen, methyl, halo and nitro, and wherein particularly preferred leaving groups are chloro, bromo, methanesulphonyloxy, and p-toluenesulphonyloxy,

including all stereoisomeric forms of said compounds, including but not limited to enantiomers and diastereoisomers as well as racemic mixtures,

and any suitable salt with an organic or inorganic acid, ester, complex or solvate thereof.

In a preferred embodiment, the invention is directed towards a compound of the general formula II, wherein

W is 2-propynyl,

A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,

R¹ is methyl,

R² being a leaving group, wherein preferred leaving groups are selected from halogen, C₁-C₆-alkylsulphonyloxy, which is optionally substituted by fluorine, and arylsulphonyloxy, which is optionally substituted by hydrogen, methyl, halo and nitro, and wherein particularly preferred leaving groups are chloro, bromo, methanesulphonyloxy, and p-toluenesulphonyloxy,

including all stereoisomeric forms of said compounds, including but not limited to enantiomers and diastereoisomers as well as racemic mixtures,

and any suitable salt with an organic or inorganic acid, ester, complex or solvate thereof.

In a more preferred embodiment, the invention is directed towards a compound of the general formula II, wherein

W is 2-propynyl,

A is phenyl,

R¹ is methyl,

R² is chloro,

including all stereoisomeric forms of said compounds, including but not limited to enantiomers and diastereoisomers as well as racemic mixtures,

and any suitable salt with an organic or inorganic acid, complex or solvate thereof.

In a second aspect the invention is directed to the targeted synthesis of compounds of the general formula II from appropriate starting materials comprising, but not limited to, alcohols of the general formulae Ia and IIa, by reacting these with suitable reagents to effect conversion of the hydroxy group displayed by compounds of the formulae Ia and IIa, into a leaving group.

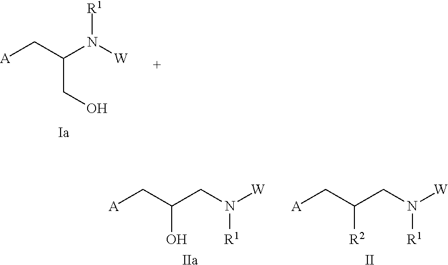

Such conversions comprise but are not limited to the reaction with a sulphonyl halide, such as methanesulphonyl chloride or p-toluenesulphonyl chloride, in the presence of a suitable base, such as a trialkyl amine, e.g. triethylamine, or such as a heteroaromatic base, e.g. 2,6-lutidine, in a suitable solvent such as an optionally halogenated hydrocarbon, e.g. dichloromethane, or an ether, such as tetrahydrofurane.

Said synthetic methods may further comprise, but are not limited to the use of sulphonyl anhydrides instead of the aforementioned sulphonyl halides, such as methanesulphonic anhydride, to give compound of the formula II in which R² is a sulphonic ester. Said synthetic methods may furthermore comprise the use of carbon tetrahalides, such as tetrachloromethane or tetrabromomethane, and suitable organophosphorus reagents such as triphenylphosphane or tri-n-butylphosphane, for the conversion of alcohols of the general formula IIa into compounds of the general formula II.

In a preferred embodiment, the invention is directed to the targeted synthesis of compounds of the general formula II from alcohols of the general formula Ia by reacting these with suitable reagents to effect conversion of the hydroxy group displayed by compounds of the formulae Ia into a leaving group. Such conversions comprise but are not limited to the reaction with a sulphonyl halide, such as methanesulphonyl chloride or p-toluenesulphonyl chloride, in the presence of a suitable base, such as a trialkyl amine, e.g. triethylamine, in a suitable solvent such as a halogenated hydrocarbon, e.g. dichloromethane, or an ether, such as tetrahydrofurane.

Said synthetic methods may further comprise, but are not limited to the use of sulphonyl anhydrides instead of the aforementioned sulphonyl halides, such as methanesulphonic anhydride, to give compound of the formula II in which R² is a sulphonic ester.

In a more preferred embodiment, the invention is directed to the targeted synthesis of compounds of the general formula II from alcohols of the general formula Ia by reacting these with suitable reagents to effect conversion of the hydroxy group displayed by compounds of the formulae Ia into a leaving group, wherein

W is 2-propynyl,

A is selected from the group comprising substituted or unsubstituted aryl, substituted or unsubstituted heteroaryl, (C₁-C₁₀-alkyl, (C₂-C₄)-alkynyl, (C₁-C₄)-alkoxy,

R¹ is methyl,

R² being a leaving group, wherein preferred leaving groups are selected from halogen, C₁-C₆-alkylsulphonyloxy, which is optionally substituted by fluorine, and arylsulphonyloxy, which is optionally substituted by hydrogen, methyl, halo and nitro, and wherein particularly preferred leaving groups are chloro, bromo, methanesulphonyloxy, and p-toluenesulphonyloxy, and wherein the most preferred leaving group is chloro,

In a more preferred embodiment, the invention is directed to the targeted synthesis of compounds of the general formula II from alcohols of the general formula Ia, wherein

W is 2-propynyl,

A is phenyl,

R¹ is methyl,

R² is chloro,

In the most preferred embodiment, the invention is directed to the targeted synthesis of compounds of the general formula II from alcohols of the general formula Ia, wherein

W is 2-propynyl,

A is phenyl,

R¹ is methyl,

R² is chloro,

by reacting said alcohol Ia with a sulphonyl chloride, such as methanesulphonyl chloride or p-toluenesulphonyl chloride, in the presence of a suitable base, such as a trialkyl amine, e.g. triethylamine, in a suitable solvent such as a halogenated hydrocarbon, e.g. dichloromethane, to effect conversion of the hydroxy group displayed by compounds of the formulae Ia into a chloro group. The reaction mixture resulting from bringing together all reactants is initially allowed to react for a suitable time ranging from 5 min to 6 hours, preferred 15 min to 4 hours, even more preferred 30 min to 2 hours, at a temperature between −50° C. and +30° C., preferred −30° C. and +30° C., even more preferred −10° C. and +25° C., followed by heating the reaction mixture for a suitable time ranging from 5 min to 6 hours, preferred 15 min to 4 hours, even more preferred 30 min to 2 hours to a temperature range between 70° C. to 130° C., preferred 80° C. to 120° C., even more preferred 90° C. to 110° C. The heating period effects the conversion of an initially formed isomeric mixture of isomers of the general formulae I and II into the desired isomer of the general formula II.

In a third aspect the invention is directed towards a method of synthesis of a compound by reacting a compound of the general formula I or II with an F-fluorinating agent, in which F═¹⁸F, to give a compound in which R² is replaced by ¹⁸F.

In a fourth aspect the invention is directed towards a method of synthesis of a compound by reacting a compound of the general formula I or II with an F-fluorinating agent, wherein said F-fluorinating agent is a compound comprising F-anions, preferably a compound selected from the group comprising 4,7,13,16,21,24-hexaoxa-1,10-diazabicyclo[8.8.8]-hexacosane K F, i.e. crown ether salt Kryptofix KF, KF, HF, KH F₂, CsF, NaF and tetraalkylammonium salts of F, such as tetrabutylammonium fluoride, and wherein F═¹⁸F, to give a compound in which R² is replaced by ¹⁸F.

In a fifth aspect, the invention is directed towards the use of the compounds of the general formulae I and II for the preparation of an ¹⁸F labelled diagnostic imaging agent or imaging agent, preferably as imaging agent for PET application.

In a more preferred embodiment, said PET application is used for imaging of CNS diseases. CNS diseases include but are not limited to inflammatory and autoimmune, allergic, infectious and toxin-triggered and ischemia-triggered diseases, pharmacologically triggered inflammation with pathophysiological relevance, neuroinflammatory, neurodegenerative diseases.

More preferably, the CNS disease is selected from multiple sclerosis, Alzheimer's disease, frontotemporal dementia, dementia with Levy bodies, leukoencephalopathy, epilepsy, neuropathic pain, amyotrophic lateral sclerosis, Parkinson's Disease, encephalopathies, brain tumors, depression, drug abuse, atheroma, atherosclerosis, pharmacologically triggered inflammation, systemic inflammation of unclear origin.

The invention also relates to kits comprising compounds of formula I or II. Such kits may contain at least one sealed vial containing a compound of formula I or II. The kit may also contain reagents suitable to perform the herein disclosed reactions. The reagents disclosed herein may be also included in such kit and may be stored in a sealed vial. The kit may also contain F-18 labelling reagents. Furthermore, the kit may contain instructions for its use.

In particular the invention relates to:

1. A compound of general formula II,

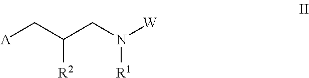

wherein

W is selected from the group comprising

—C(U¹)(U²)—C≡CH and cyclopropyl, U¹ and U² being independently selected from hydrogen and deuterium,

A is selected from the group comprising substituted or unsubstituted aryl, substituted or unsubstituted heteroaryl, (C₁-C₁₀-alkyl, (C₂-C₄)-alkynyl, (C₁-C₄)-alkoxy,

R¹ is selected from (C₁-C₆)-alkyl,

R² is a leaving group,

including all stereoisomeric forms of said compounds,

and any suitable salt with an organic or inorganic acid, ester, complex or solvate thereof.

2. A compound of general formula II,

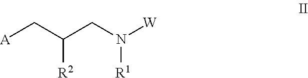

wherein

W is selected from the group comprising

—C(U¹)(U²)—C≡CH and cyclopropyl, U¹ and U² being independently selected from hydrogen and deuterium,

A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,

R¹ is selected from (C₁-C₆)-alkyl,

R² is a leaving group.

3. A compound according to count 1 or 2, wherein

R² is chloro.

4. A method of targeted synthesis of a compound of count 1 or 2,

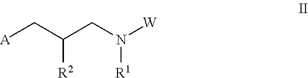

including reacting a compound of formula Ia or IIa,

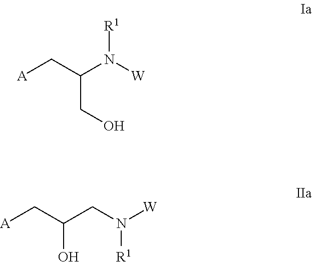

with suitable reagents to effect conversion of the hydroxyl group of formula Ia or IIa into a leaving group,

wherein W is selected from the group comprising

—C(U¹)(U²)—C≡CH and cyclopropyl, U¹ and U² being independently selected from hydrogen and deuterium,

A is selected from the group comprising substituted or unsubstituted aryl, substituted or unsubstituted heteroaryl, (C₁-C₁₀-alkyl, (C₂-C₄)-alkynyl, (C₁-C₄)-alkoxy,

R¹ is selected from (C₁-C₆)-alkyl,

R² is a leaving group.

5. A method according to count 4,

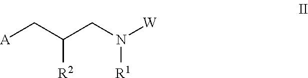

including reacting a compound of formula Ia

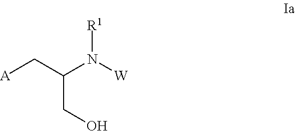

with suitable reagents to effect conversion of the hydroxyl group of formula Ia or IIa into a leaving group,

wherein W is selected from the group comprising

—C(U¹)(U²)—C≡CH and cyclopropyl, U¹ and U² being independently selected from hydrogen and deuterium;

A is selected from the group comprising substituted or unsubstituted aryl, substituted or unsubstituted heteroaryl, (C₁-C₁₀-alkyl, (C₂-C₄)-alkynyl, (C₁-C₄)-alkoxy,

R¹ is selected from (C₁-C₆)-alkyl,

R² being a leaving group,

6. A method according to count 4 or 5, wherein

W is 2-propynyl,

A is phenyl,

R¹ is methyl,

R² is chloro.

7. A method according to count 6, wherein

W is 2-propynyl,

A is phenyl,

R¹ is methyl,

R² is chloro,

and wherein the reaction mixture is heated to a temperature between 70° C.-130° C. after the reaction mixture was incubated at a lower temperature.

8. A method of synthesis of a compound of formula If

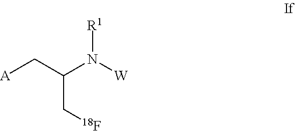

by reacting a compound of formula II

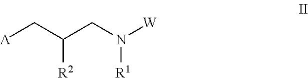

with an F-fluorinating agent, wherein F═F-18,

wherein

W is selected from the group comprising

—C(U¹)(U²)—C≡CH and cyclopropyl, U¹ and U² being independently selected from hydrogen and deuterium;

A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,

R¹ is selected from (C₁-C₆)-alkyl,

R² is a leaving group.

9. A method according to count 8, wherein

W is 2-propynyl,

A is phenyl,

R¹ is methyl,

R² is chloro,

10. A kit comprising a sealed vial containing a compound according to count 1, 2, or 3.

Definitions

As used herein, a leaving group refers to a functional group selected from the group comprising halo, in particular chloro, bromo, iodo, or an optionally substituted sulphonyloxy group, such as methanesulphonyloxy, p-toluenesulphonyloxy, trifluoromethanesulphonyloxy, nonafluorobutanesulphonyloxy, (4-bromo-benzene)sulphonyloxy, (4-nitro-benzene)sulphonyloxy, (2-nitro-benzene)sulphonyloxy, (4-isopropyl-benzene)sulphonyloxy, (2,4,6-tri-isopropyl-benzene)sulphonyloxy, (2,4,6-trimethyl-benzene)sulphonyloxy, (4-tertbutyl-benzene)sulphonyloxy, benzenesulphonyloxy, and (4-methoxy-benzene)sulphonyloxy.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl, which themselves can be substituted with one, two or three substituents independently and individually selected from the group comprising halo, nitro, (C₁-C₆)-alkylcarbonyl, cyano, nitrile, hydroxyl, trifluoromethyl, (C₁-C₆)-alkylsulphonyl, (C₁-C₆)-alkyl, (C₁-C₆)-alkoxy and (C₁-C₆)-alkylsulphanyl. As outlined above such “aryl” may additionally be substituted by one or several substituents.

The term “heteroaryl” as employed herein refers to groups having 5 to 14 ring atoms; 6, 10 or 14 π (pi) electrons shared in a cyclic array; and containing carbon atoms (which can be substituted with halo, nitro, (C₁-C₆)-alkylcarbonyl, cyano, nitrile, trifluoromethyl, (C₁-C₆)-alkylsulphonyl, (C₁-C₆)-alkyl, (C₁-C₆)-alkoxy or (C₁-C₆)-alkylsulphanyl) and 1, 2, 3 or 4 oxygen, nitrogen or sulphur heteroatoms (where examples of heteroaryl groups are: thienyl, benzo[b]thienyl, naphtho[2,3-b]thienyl, thianthrenyl, furyl, furanyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl groups).

Heteroaryl can be substituted with one, two or three substituents independently and individually selected from the group comprising halo, nitro, (C₁-C₆)-alkylcarbonyl, cyano, nitrile, hydroxyl, trifluoromethyl, (C₁-C₆)-alkylsulphonyl, (C₁-C₆)-alkyl, (C₁-C₆)-alkoxy and (C₁-C₆)-alkylsulphanyl. As outlined above such “heteroaryl” may additionally be substituted by one or several substituents.

As used herein in the description of the invention and in the claims, the term “alkyl”, by itself or as part of another group, refers to a straight chain or branched chain alkyl group with 1 to 10 carbon atoms such as, for example methyl, ethyl, propyl, isopropyl, butyl, iso-butyl, tert-butyl, pentyl, iso-pentyl, neopentyl, heptyl, hexyl, decyl. Alkyl groups can also be substituted, such as by halogen atoms, hydroxyl groups, C₁-C₄ alkoxy groups or C₆-C₁₂ aryl groups (which, in turn, can themselves be substituted, such as by 1 to 3 halogen atoms). More preferably alkyl is C₁-C₁₀ alkyl, C₁-C₆ alkyl or C₁-C₄ alkyl.

As used herein in the description of the invention and in the claims, the term alkynyl is similarly defined as for alkyl, but is meant to contain at least one carbon-carbon double or triple bond, respectively, more preferably C₃-C₄ alkynyl.

As used herein in the description of the invention and in the claims, the term “alkoxy (or alkyloxy)” refer to alkyl groups respectively linked by an oxygen atom, with the alkyl portion being as defined above.

Whenever the term “substituted” is used, it is meant to indicate that one or more hydrogens attached to the atom indicated in the expression using “substituted” is/are replaced with a selection from the indicated group of substituents, provided that the indicated atom's normal valency is not exceeded, and that the substitution results in a chemically stable compound, i. e. a compound that is sufficiently robust to survive isolation to a useful degree of purity from a reaction mixture, and formulation into a pharmaceutical composition. The substituent groups may be selected from halogen atoms, hydroxyl groups, nitro, (C₁-C₆)-alkylcarbonyl, cyano, nitrile, trifluoromethyl, (C₁-C₆)-alkylsulphonyl, (C₁-C₆)-alkyl, (C₁-C₆)-alkoxy and (C₁-C₆)-alkylsulphanyl.

As used herein in the description of the invention and in the claims, the terms “inorganic acid” and “organic acid”, refer to mineral acids, including, but not being limited to: acids such as carbonic, nitric, phosphoric, hydrochloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulphonic, ethanesulphonic, benzenesulphonic, phantothenic, toluenesulphonic, trifluormethansulphonic and sulphanilic acid, respectively.

The compounds of the present invention can exist as solvates, such as hydrates, wherein compounds of the present invention may contain organic solvents or water as structural element of the crystal lattice of the compounds. The amount of said solvents may exist in a stoichiometric or unstoichiometric ratio. In case of stoichiometric solvates, e.g. hydrates, hemi-, (semi-), mono-, sesqui-, di-, tri-, tetra-, penta- etc. solvates or hydrates are possible.

If a chiral centre or another form of an isomeric centre is present in a compound according to the present invention, all forms of such isomers, including enantiomers and diastereoisomers, are intended to be covered herein. Compounds containing a chiral centre may be used as racemic mixture or as an enantiomerically enriched mixture, or the racemic mixture may be separated using well-known techniques and a single enantiomer may be used. In cases in which compounds have unsaturated double bonds, both the E- and Z-isomer are within the scope of this invention. In cases wherein compounds may exist in tautomeric forms, such as keto-enol tautomers, each tautomeric form is contemplated as being included within this invention whether existing in equilibrium or predominantly in one form.

The terms “halogen”, or “halo” refers to fluorine (F), chlorine (Cl), bromine (Br), or iodine (I); the term “halide” refers to fluoride, chloride, bromide or iodide.

General Synthesis of Compounds of the Invention

Compounds of the invention can be prepared readily by various methods, inter alia from alcohols of the general formulae Ia and IIa. Such compounds can be approached starting from well-known and/or commercially available starting materials, and using synthetic methods well known to the person skilled in the art.

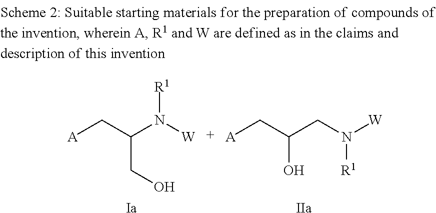

Thus, N-alkyl amino acids of the general formula III can be reduced using complex hydride reagents, such as lithium aluminum hydride, to give the respective amino alcohols IV, which can be converted to intermediates of the general formula Ia by alkylation or propargylation employing reagents of the general formula W—R², such as propargyl bromide. Alternatively, the elaboration of IV to Ib might be accomplished by Mitsunobu-type coupling reactions, employing IV, W—OH, appropriate phosphane reagents such as triphenyl phosphane or tri-n-butylphosphane, and an appropriate diazodicarboxylate, such as diethyl diazocarboxylate. Due to the widespread availability of amino acids in both enantiomeric forms, the methodology described herein offers the opportunity to approach either enantiomeric form of Ia selectively.

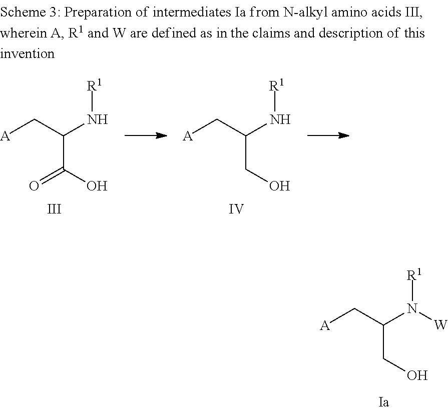

Alcohols of the general structure IIa can be, for example, approached starting from epoxides of the general formula V. Such compounds are well known to the person skilled in the art, partially available from commercial vendors, and readily accessible e.g. by epoxidation of the respective terminal alkenes. Such epoxides V can be opened by amines R¹—NH—W to give the desired aminoalcohols of the general formula IIa (see e.g. H. Lindsay et al., Synthesis 2007, (6), 902). Single enantiomers can be either obtained by the use of enantiopure epoxides as starting materials, or by resolution of enantiomers on the amine step e.g. by chiral HPLC separation or by selective crystallisation of salts formed by exposure of said amino alcohols IIa to enantiopure acids.

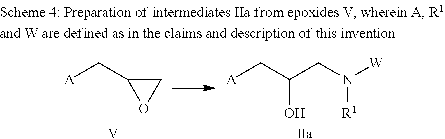

Compounds according to the formulae Ia and IIa can be transformed into the compounds of the invention inter alia by reaction with a sulphonyl chloride, such as methanesulphonyl chloride, in the presence of an appropriate base, such as a tertiary aliphatic amine, e.g. triethylamine or Huenig's base, in an appropriate solvent, such as dichloromethane.

In accordance with earlier findings published in the literature (see e.g. P. Gmeiner et al., J. Org. Chem. 1994, 59, 6676) it is assumed that initial sulphonylation e.g. of Ia resulting in sulphonate VI is followed by the formation of an aziridinium ion VII which, in turn, is opened by the chloride counterion formed in the initial reaction step. Under kinetic control, formation of the primary halide Ib via attack of the aziridinium ring carbon lacking a substituent by the halide counterion would be favoured. Thermodynamic control, in turn, would favour the formation of the secondary halide IIb. Whilst Gmeiner's publication reports the formation of a related halide featuring bulky N,N dibenzyl substitution already at room temperature, we were surprised to find that elevated temperature was required for certain compounds of the invention to effect rearrangement towards the secondary halide IIb.

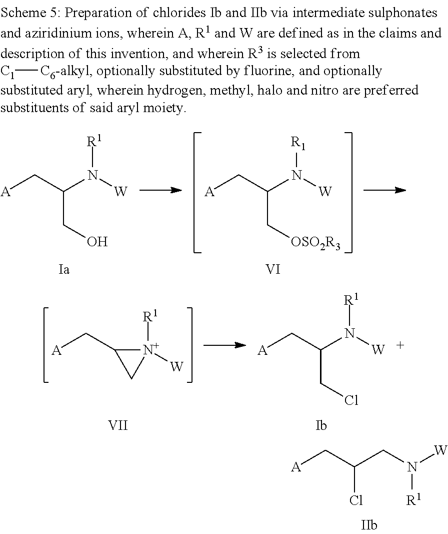

Alternatively, formation of compounds of the general formulae I and, more preferably, II, can be accomplished by reaction of suitable starting materials, such as alcohols Ia and Ib, with sulphonic acid anhydrates (to furnish the respective sulphonates), or with the respective carbon tetrahalides, such as carbon tetrachloride, and appropriate phosphorus reagents such as triphenyl phosphane (see e.g. R. Appel et al, Angew. Chem. Int. Ed. Engl. 1975, 14, 801).

### DESCRIPTION OF THE FIGURES

FIG. 1 shows the chiral HPLC of Intermediate 1B.

FIG. 2 shows the chiral HPLC of the optical antipode of Intermediate 1B.

FIG. 3 shows the chiral HPLC of Example 1

FIG. 4 shows the chiral HPLC of the optical antipode of Example 1.

FIG. 5 shows the preparative HPLC of Example 4.

FIG. 6 shows the co-injection of the desired F-18 labelled product of Example 4 with its non-radioactive reference compound in chiral HPLC.

FIG. 7 shows the co-injection of the desired F-18 labelled product of Example 4 with its non-radioactive reference compound's optical antipode in chiral HPLC.

FIG. 8 shows the co-injection of the F-18 labelled by-product of Example 4 with its non-radioactive reference compound in chiral HPLC.

### EXPERIMENTAL SECTION

General: All solvents and chemicals were obtained from commercial sources and used without further purification. The following table lists the abbreviations used in this paragraph and in the Examples section as far as they are not explained within the text body. NMR peak forms are stated as they appear in the spectra, possible higher order effects have not been considered.

Reactions employing microwave irradiation can be run with a Biotage Initiator® microwave optionally oven equipped with a robotic unit. The compounds and intermediates produced according to the methods of the invention may require purification. Purification of organic compounds is well known to the person skilled in the art and there may be several ways of purifying the same compound. In some cases, no purification may be necessary. In certain cases, the compounds may be purified by crystallization. In some cases, impurities may be removed by trituration using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with e.g. a FlashMaster II autopurifier (Argonaut/Biotage) and eluents such as gradients of hexane/EtOAc or dichloromethane/ethanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid or aqueous ammonia. In some cases, purification methods as described above can provide those compounds of the present invention which possess a sufficiently basic functionality in the form of a salt, such as, in the case of a compound of the present invention which is sufficiently basic, a trifluoroacetate or formate salt for example. A salt of this type may be transformed into its free base form, respectively, by various methods known to the persion skilled in the art.

Abbreviations

| | |
|---|---|
| Br | Broad signal (in NMR) |
| D | Doublet |
| Dd | Doublet of doublet |
| DMSO | Dimethylsulphoxide |
| Ee | Enantiomeric excess |
| ESI | Electrospray ionisation |
| EtOAc | Ethyl acetate |
| K_{2.2.2} | 4, 7, 13, 16, 21, 24-hexaoxa-1,10- |
| | diazabicyclo[8.8.8]-hexacosane |
| MS | Mass spectrometry |
| MTB | Methyl tert-butyl ether |
| M | Multiplet |
| NMR | Nuclear magnetic resonance |
| | spectroscopy: chemical shifts (δ) are |
| | given in ppm. |
| RT | Room temperature |
| S | Singlet |
| T | Triplet |
| THF | Tetrahydrofurane |

### Intermediate 1A: (2S)-2-(methylamino)-3-phenylpropan-1-ol

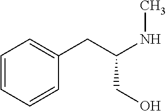

To a suspension of N-methyl-L-phenylalanine (20 g, 112 mmol) in THF (1200 mL) cooled to −10° C. was added in small portions lithium aluminum hydride (6.35 g, 167 mmol). After ceasing of the initial exothermic reaction, the cooling bath was removed and the reaction mixture was heated at reflux overnight. Subsequently, another portion of lithium aluminum hydride (4.24 g, 112 mmol) was added after cooling to −10° C., followed by refluxing for an additional 3 hours. The reaction mixture was cooled to −40° C., and aqueous 2 N sodium hydroxide was added cautiously. After warming up to RT, the mixture was filtered, the residue was washed with MTB, and the filtrate was evaporated to give the crude target compound (17.7 g, 96% yield) which was used without further purification.

¹H NMR (400 MHz, DMSO-d₆) δ ppm 2.26 (s, 3H), 2.51-2.62 (m, 3H) 3.16-3.30 (m, 3H), 7.11-7.25 (m, 5H).

MS (ESI): [M+H]⁺=166.

### Intermediate 1B: (2S)-2-[methyl(prop-2-yn-1-yl)amino]-3-phenylpropan-1-ol

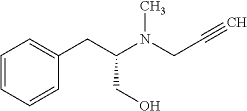

To a solution of (2S)-2-(methylamino)-3-phenylpropan-1-ol (17.7 g, 107 mmol) in THF (355 mL) was added potassium carbonate (70.8 g, 512 mmol) at RT. After stirring the resulting mixture for 30 min, 3-bromo propyne (9.32 mL, 124 mmol) was added, followed by stirring for 60 h at RT. Water (1300 mL) was added, the organic layer was separated and the aqueous layer was extracted with dichloromethane (3×500 mL). The combined aqueous layers were washed with aqueous sodium bicarbonate, dried over sodium sulphate, and evaporated. The crude product was purified by column chromatography on silica gel (gradient hexane→hexane/EtOAc 1:3) to give the desired product (10.3 g, 47% yield).

¹H NMR (400 MHz, CHLOROFORM-d) δ ppm 2.29 (t, 1H) 2.34-2.46 (m, 1H) 2.43 (s, 3H) 2.83-2.95 (s br, 1H) 3.03-3.13 (m, 2H) 3.32-3.38 (m, 1H) 3.39-3.47 (m, 3H) 7.15-7.32 (m, 5H).

MS (ESI): [M+H]⁺=204.

### EXAMPLE 1

### N-[(2R)-2-chloro-3-phenylpropyl]-N-methylprop-2-yn-1-amine

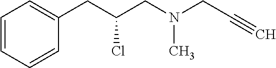

To a solution of (2S)-2-[methyl(prop-2-yn-1-yl)amino]-3-phenylpropan-1-ol (200 mg, 0.98 mmol) in dichloromethane (10 mL) was added triethylamine (206 μL, 1.48 mmol), and the mixture was cooled to 0° C. Methanesulphonyl chloride (99 μL, 1.28 mmol) was added, and the cooling bath was removed. After stirring at RT for 1 h, the reaction mixture was heated to 100° C. in a microwave oven for 1 h. After cooling to RT, the mixture was extracted by aqueous sodium bicarbonate, followed by brine. The organic layer was dried over sodium sulphate and evaporated. Column chromatography on silica gel (EtOAc in hexane 2%→16%) gave the title compound containing only traces of the corresponding primary regioisomer (140 mg, 64% yield).

¹H NMR (400 MHz, CHLOROFORM-d) δ ppm 2.21 (t, 1H) 2.38 (s, 3H) 2.74 (d, 2H) 2.94 (dd, 1H) 3.23 (dd, 1H) 3.43 (dd, 2H) 4.10-4.18 (m, 1H) 7.22-7.35 (m, 5H).

MS (ESI): [M+H]⁺=222.

### EXAMPLE 2

### Analytical Documentation of the Stereospecifity of the Transformation of Intermediate 1B into Compound Example1

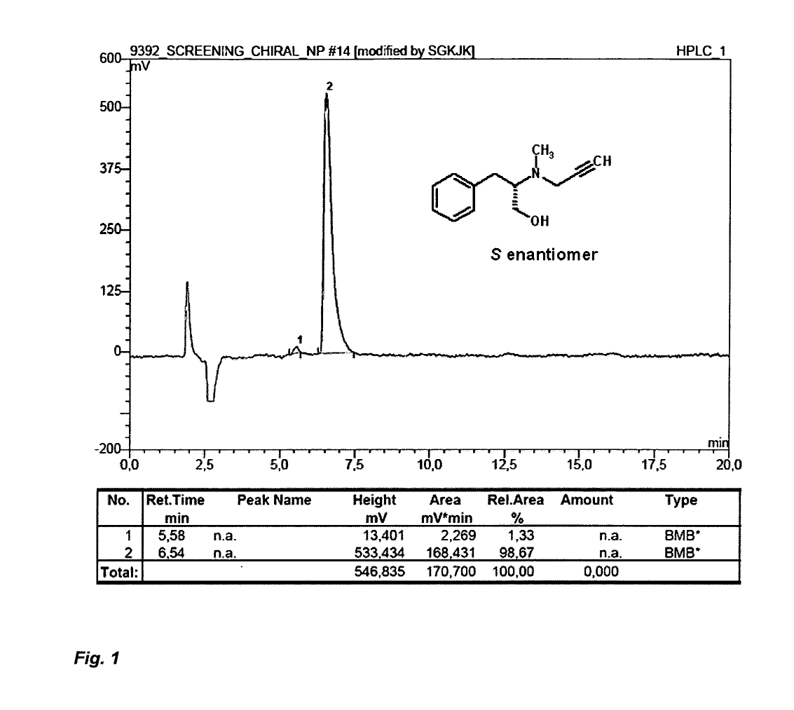

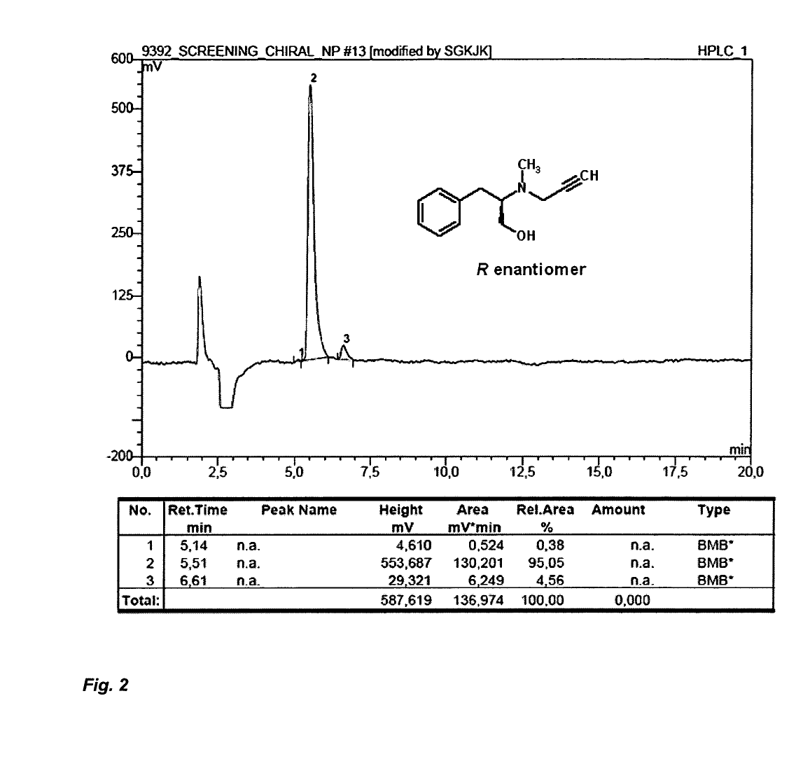

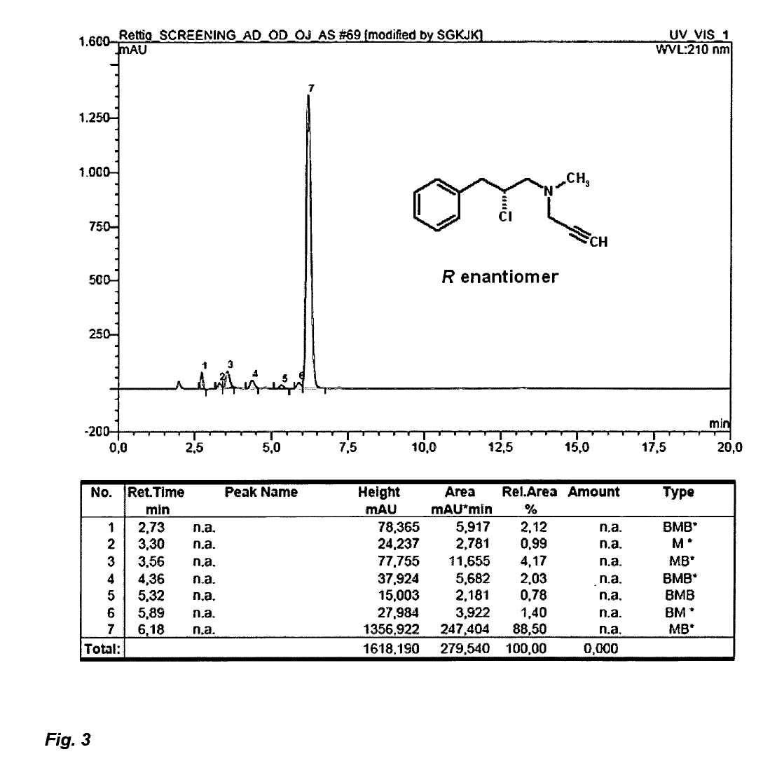

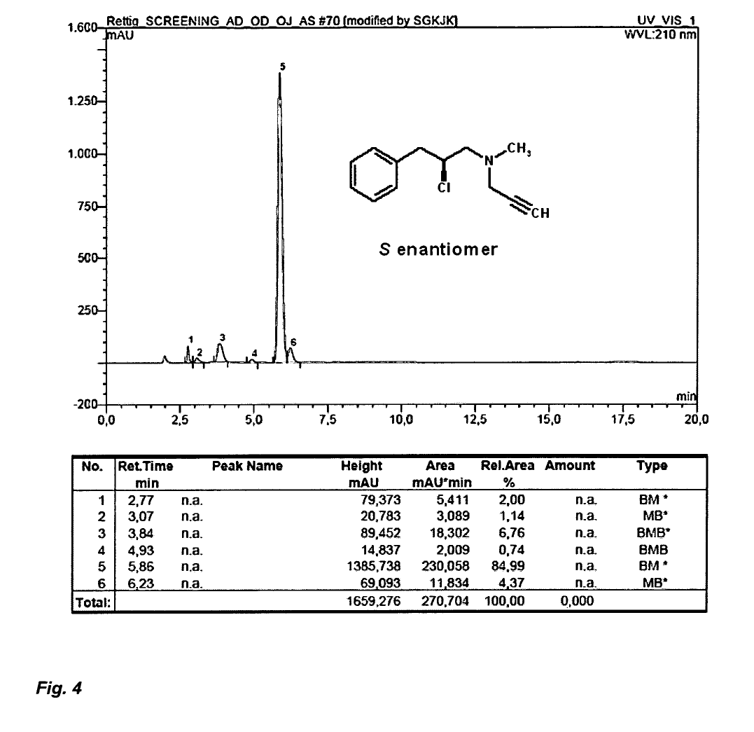

This was achieved by preparing Intermediate 1B and its optical antipode, (2R)-2-[methyl(prop-2-yn-1-yl)amino]-3-phenylpropan-1-ol, determination of their ee by means of chiral HPLC (see FIGS. 1 and 2), and subjecting both alcohols independently from each other to the synthetic protocol described for the preparation of Example 1, followed by determination of the ee of the resulting chlorides, i.e. Example 1 and its optical antipode (see FIGS. 3 and 4). It is worth noting that the absolute configuration of Example 1 has been assigned according to literature studies, see e.g. P. Gmeiner et al., J. Org. Chem. 1994, 59, 6676, or J. Cossy et al., Chem. Eur. J. 2009, 15, 1064).

Analytical chiral HPLC of Intermediate 1B and its optical antipode has been performed using a Chiralpak IA 5μ 150×4.6 mm column. As eluent, an isocratic 90/10 mixture of hexane/ethanol containing 0.1% diethylamine as a buffer was used.

| | Retention time (min) | ee (%) |
|---|---|---|
| Intermediate 1B | 6.5 | 97 |
| Optical antipode thereof | 5.5 | 91 |

Analytical chiral HPLC of Example 1 and its optical antipode has been performed using a Chiralcel OJ-H 5μ 150×4.6 mm column and an isocratic 95/5 mixture of hexane/iso-propanol as an eluent.

| | Retention time (min) | ee (%) |
|---|---|---|
| Example 1 | 6.2 | 97 |
| Optical antipode thereof | 5.9 | 90 |

### EXAMPLE 3

### [¹⁸F]-Fluorination of N-[(2R)-2-chloro-3-phenylpropyl]-N-methylprop-2-yn-1-amine

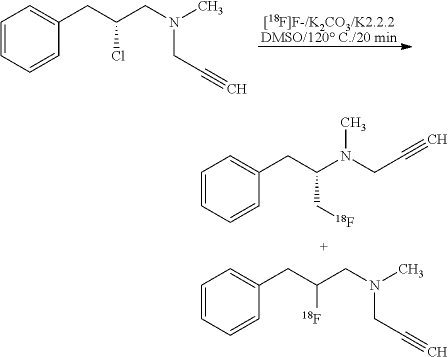

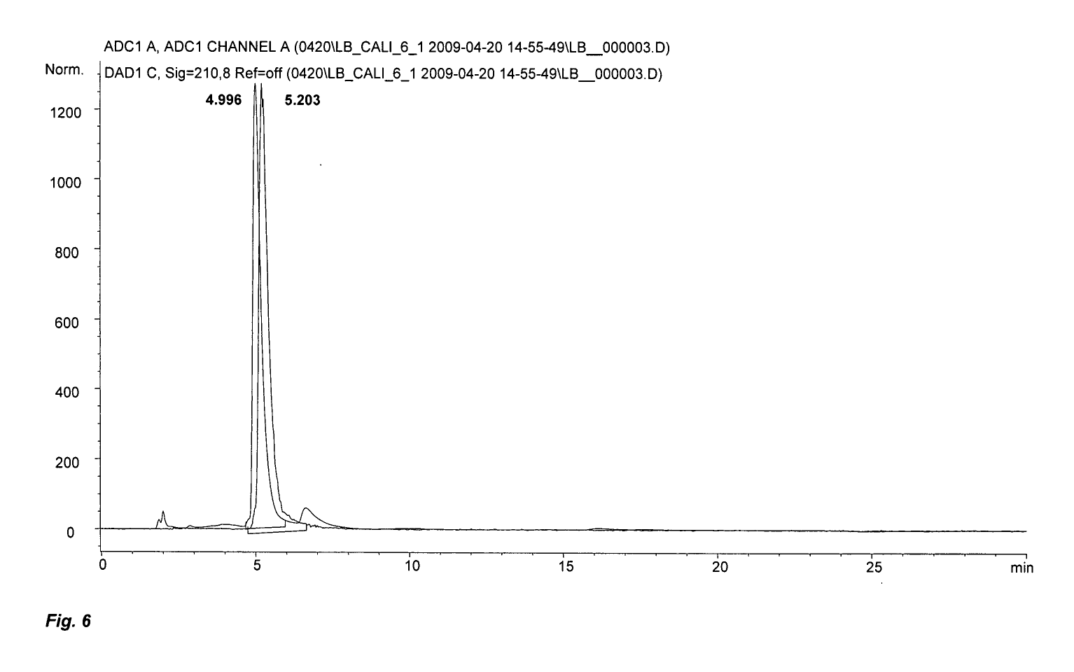

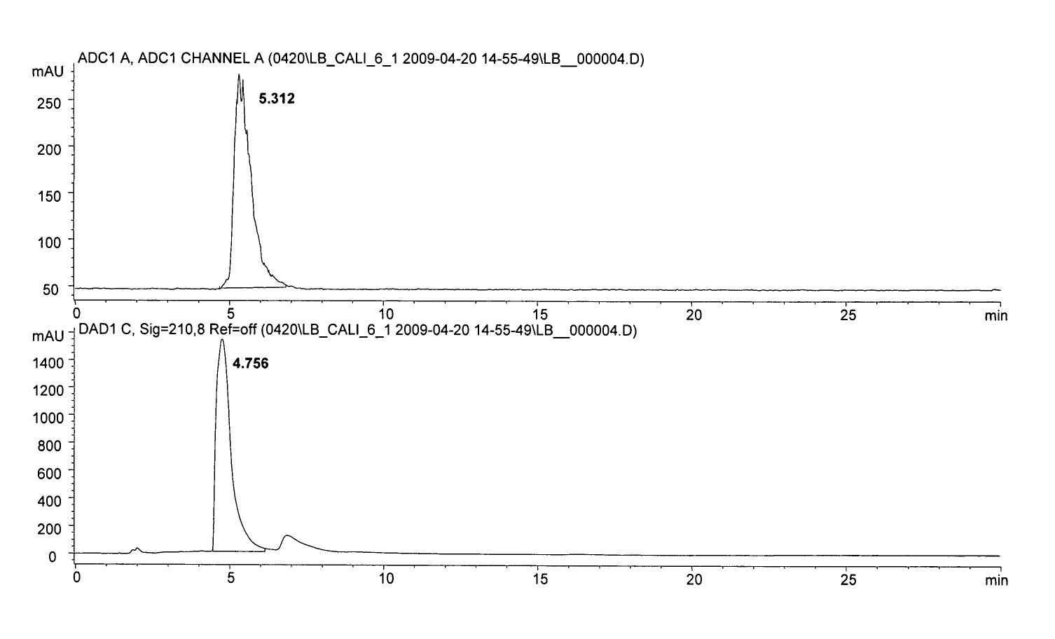

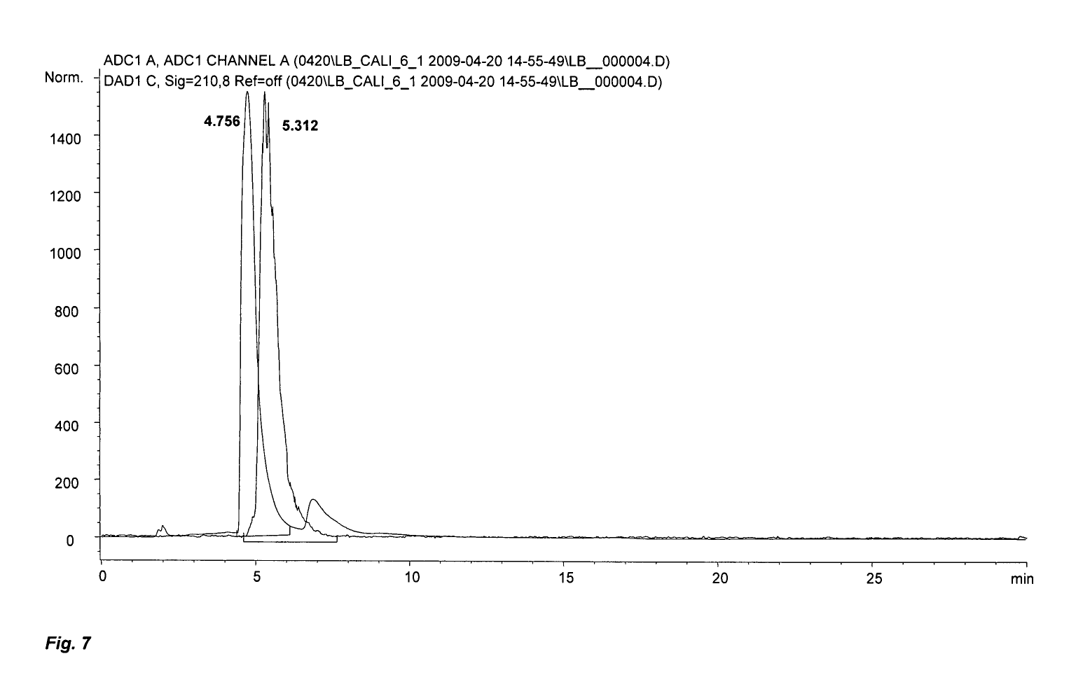

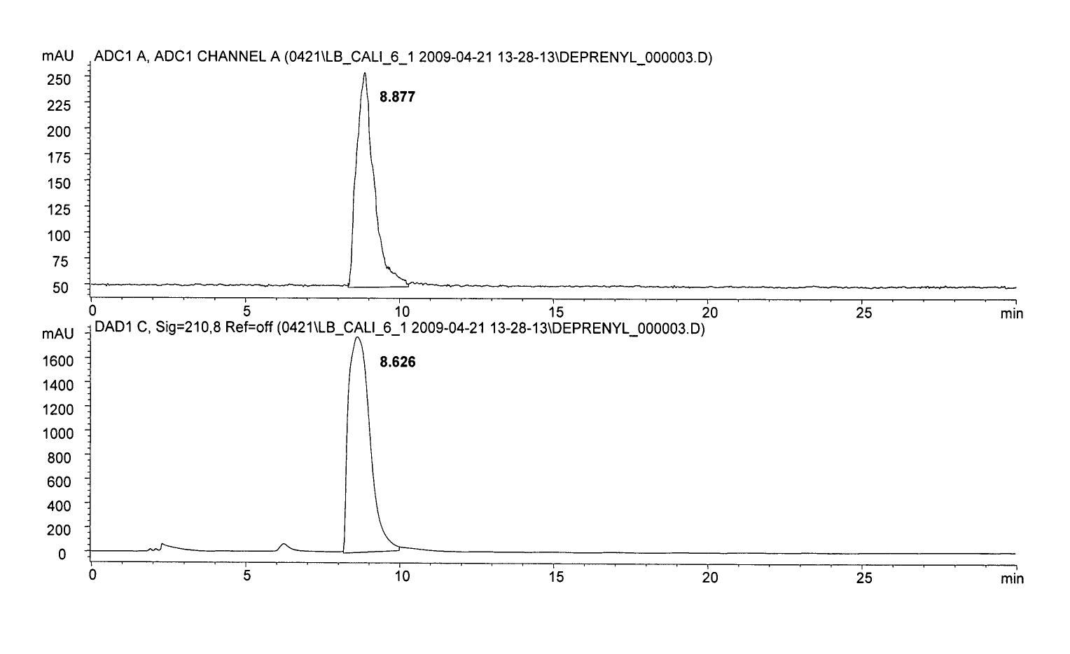

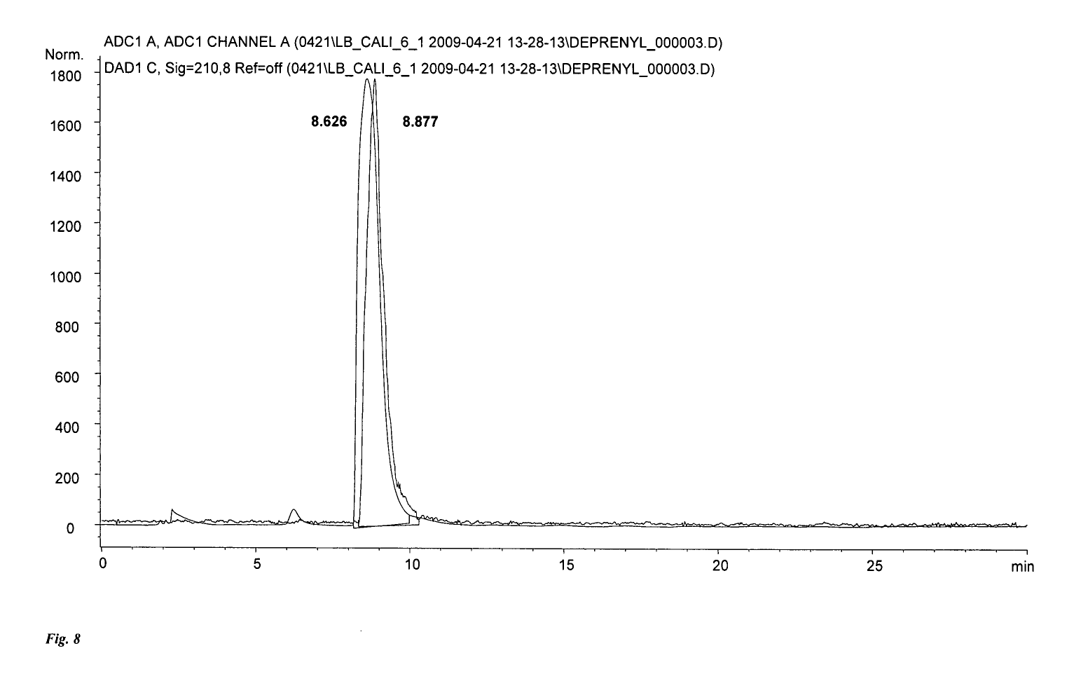

Aqueous [¹⁸F]Fluoride (0.9 GBq) was trapped on a QMA cartridge (Waters, Sep Pak Light QMA Part. No.: WAT023525) and eluted with 5 mg K_{2.2.2} in 0.95 mL acetonitrile+1 mg potassium carbonate in 50 μL water into a Wheaton vial (5 mL). The solvent was removed by heating at 120° C. for 10 min under a stream of nitrogen. Anhydrous acetonitrile (1 mL) was added and evaporated as before. A solution of precursor (Example 1) (2 mg) in 500 μL anhydrous DMSO was added. After heating at 120° C. for 20 min the crude reaction mixture was diluted with water to a total volume of 5 mL and purified by preparative HPLC (see FIG. 3): ACE 5-C18-HL 250 mm×10 mm, Advanced Chromatography Technologies; Cat. No.: ACE 321-2510; 0.01 M phosphoric acid/acetonitrile (85:15), isocratic, flow: 4 mL/min. The collected HPLC fraction (t_{R}=18.8 min) was diluted with 40 mL water and immobilized on a Sep-Pak light C18 cartridge (Waters, WAT023501), which was washed with 5 mL water and eluted with 1 mL ethanol to deliver 86 MBq of the product (18%, corrected for decay; radiochemical purity>99%). The radioactive products were analysed by chiral HPLC (Chiralcel OJ-H 5 μm 150×4.6; A): hexane, B): ethanol, 30 min, 1% B; isocratic; 1 mL/min), and showed co-elution with the respective ¹⁹F standards which are accessible to the person skilled in the art by application of standard fluorination methods well known in the art on compounds such as Intermediate 1B (FIG. 4 and FIG. 6).

Analytical chiral HPLC of desired F-18 labelled isomer as prepared in Example 3 (t_{R}=5.2 min) shows co-elution nicely with the non-radioactive reference compound (t_{R}=4.99 min) (see FIG. 4).

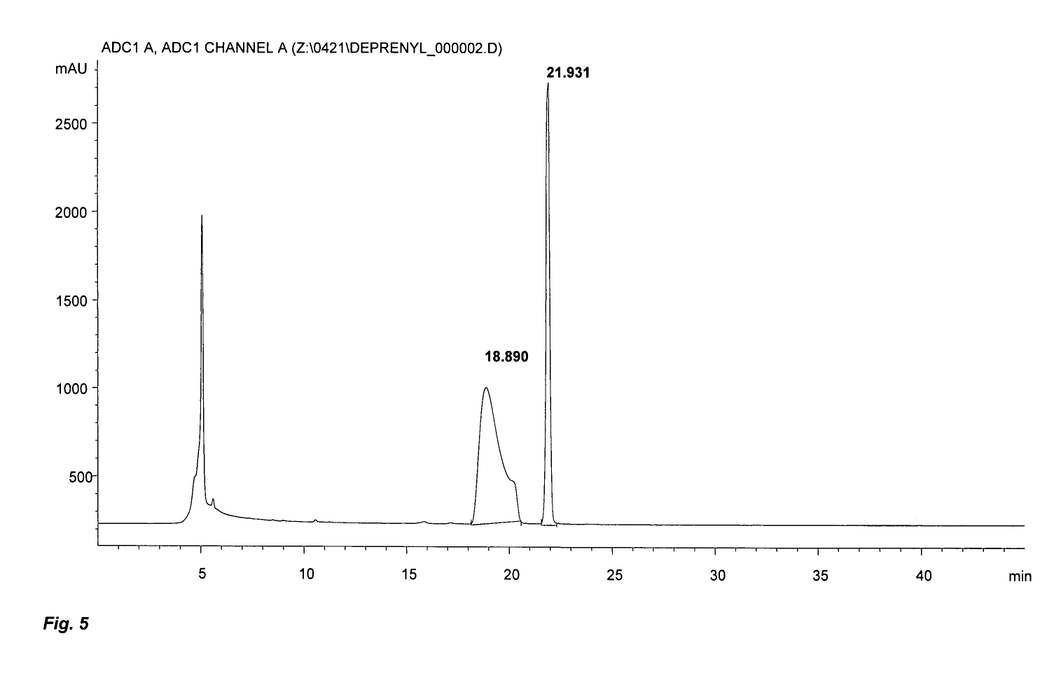

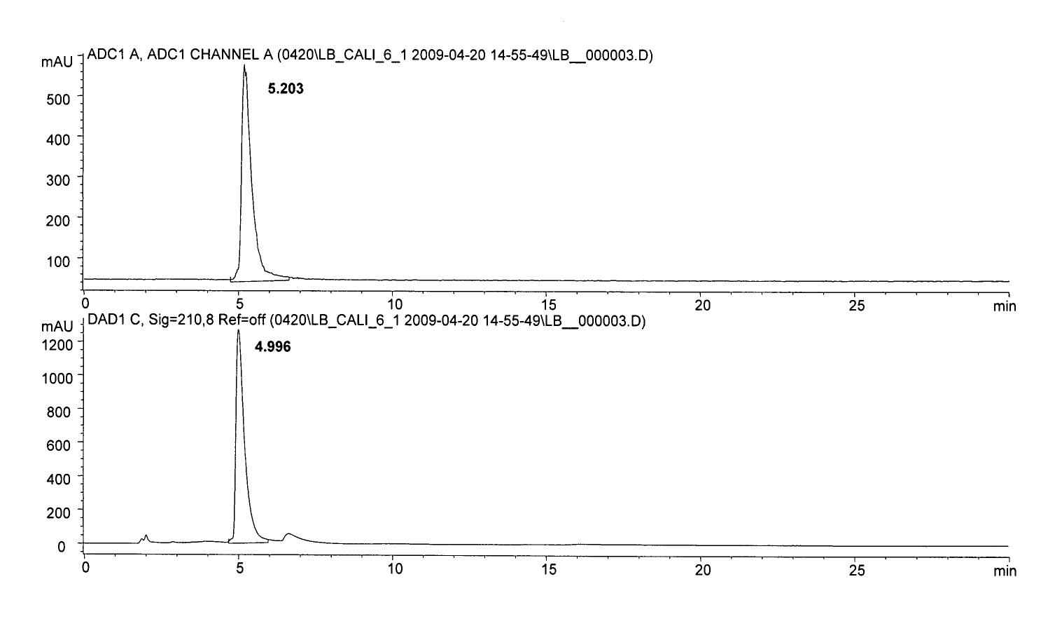

Analytical chiral HPLC of desired F-18 labelled isomer as prepared in Example 3 (t_{R}=5.3 min) shows clearly that the optical antipode of its non-radioactive F-19 reference elutes differently (t_{R}=4.75 min) (see FIG. 5).

The radioactive by-product at t_{R}=21.9 min (FIG. 3) was collected as well and also characterized by chiral HPLC (t_{R}=8.8 min). It shows co-elution with its non radioactive reference compound (t_{R}=8.6 min) (see FIG. 6).

1. A compound of general formula II, 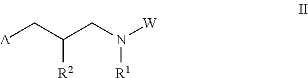
wherein
W is selected from the group comprising
—C(U¹)(U²)—C≡CH and cyclopropyl, U¹ and U² being independently selected from hydrogen and deuterium;
A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,
R¹ is selected from (C₁-C₆)-alkyl,
R² being a leaving group,
including all stereoisomeric forms of said compounds,
and any suitable salt with an organic or inorganic acid, ester, complex or solvate thereof.

2. A compound according to claim 1, wherein W is 2-propynyl.

3. A compound according to claim 1, wherein R¹ is methyl.

4. A compound according to claim 1, wherein R² is Cl.

5. A compound according to claim 1, wherein A is phenyl.

6. A compound according to claim 1, wherein W is 2-propynyl, R¹ is methyl, R² is Cl, and A is phenyl.

7. A method of targeted synthesis of a compound of claim 1, 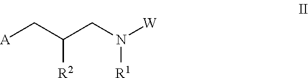
including reacting a compound of formula Ia or IIa, 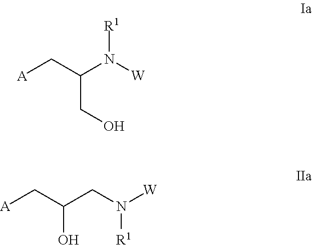
with suitable reagents to effect conversion of the hydroxyl group of formula Ia or IIa into a leaving group,
wherein W is selected from the group comprising
—C(U¹)(U²)—C≡CH and cyclopropyl, U¹ and U² being independently selected from hydrogen and deuterium;
A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,
R¹ is selected from (C₁-C₆)-alkyl,
R² being a leaving group.

8. A method according to claim 7,
wherein W is 2-propynyl.

9. A method according to claim 7,
wherein A is phenyl.

10. A method according to claim 7,
wherein R¹ is methyl.

11. A method according to claim 7,
wherein R² is Cl.

12. A method according to claim 7,
wherein W is 2-propynyl, R¹ is methyl, R² is Cl, and A is phenyl.

13. A method of synthesis of a compound of formula If 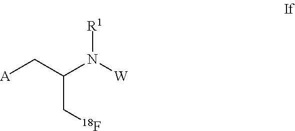
by reacting a compound of formula II 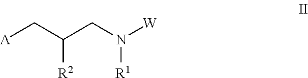
with an F-fluorinating agent, wherein F═F-18,
wherein
W is selected from the group comprising
—C(U¹)(U²)—C≡CH and cyclopropyl, U¹ and U² being independently selected from hydrogen and deuterium,
A is selected from the group comprising substituted or unsubstituted aryl, substituted or unsubstituted heteroaryl, (C₁-C₁₀)-alkyl, (C₂-C₄)-alkynyl, (C₁-C₄)-alkoxy,
R¹ is selected from (C₁-C₆)-alkyl,
R² being a leaving group.

14. A method according to claim 13,
wherein W is 2-propynyl.

15. A method according to claim 13,
wherein A is phenyl.

16. A method according to claim 13,
wherein R¹ is methyl.

17. A method according to claim 13,
wherein R² is Cl.

18. A method according to claim 13,
wherein W is 2-propynyl, is methyl, R² is Cl, R¹ is methyl and A is phenyl.

19. A method according to claim 12, wherein the reaction mixture is heated to a temperature between 70° C.-130° C. after the reaction mixture was incubated at a lower temperature.

20. A kit comprising a sealed vial containing a compound according to claim 1.
